(19) United States
(12) Patent Application Publication
(10) Pub. No.: US 2002/0071850 A1
(43) Pub. Date: Jun. 13, 2002

(54) HELICOBACTER PYLORI ANTIGEN

(76) Inventors: CHRISTOPHER JOHN SMITH, DEESIDE (GB); ROBERT LLEWELLYN CLANCY, NEWCASTLE (AU); ALLAN WILLIAM CRIPPS, FARRER (AU)

Correspondence Address: FOLEY & LARDNER, 3000 K STREET N W, SUITE 500, P O BOX 25696, WASHINGTON, DC, 200078696

(21) Appl. No.: 09/383,387

(22) Filed: Aug. 26, 1999

Related U.S. Application Data
(63) Continuation-in-part of application No. 09/165,895, filed on Sep. 30, 1998, now abandoned.

(30) Foreign Application Priority Data
Nov. 15, 1994 (GB) 944 22 990

(51) Int. Cl. A61K 39/02

(52) U.S. Cl. 424/234.1

(57) ABSTRACT

A novel protein extracted from H. pylori has a molecular weight of about 50-65 kDa and an amino terminal amino acid sequence that has at least 60% identity to a sequence selected from the group consisting of:

M V T L I N N E D D
Met-Val-Thr-Leu-Ile-Asn-Asn-Glu-Asp-Asp; and
S V T L I N N E N N E R
Ser-Val-Thr-Leu-Ile-Asn-Asn-Glu-Asn-Asn-Glu-Arg-
Y Y F E T
Tyr-Tyr-phe-Glu-Thr.

The protein is capable of inducing anti H. pylori antibodies and can be used as a vaccine against H. pylori.

### FIELD OF THE INVENTION

The invention relates to a novel antigen of Helicobacter pylori, or antigenic fragments thereof, the use of the antigen or fragments thereof in detecting Helicobacter pylori and kits comprising them as well as vaccines comprising the antigen or fragments thereof and a method for the isolation of the antigen.

### BACKGROUND OF THE INVENTION

Gut infections in mammals, and in particular humans, stimulate an immune response in mucous secretions, such as saliva, through activation of the common mucosal immune system. This response often initially parallels an antibody response in serum although is generally characterised by the presence of IgA antibodies. However, the immune response in secretions, including saliva, rapidly diminishes following elimination of the antigen (eg bacteria or virus) from the body. Accordingly, the presence of antibody in mucous secretions reflects current, i.e. contemporary, infection. In the case of a microbial infection, for example, antibodies in mucous secretions, herein after referred to as secretious antibodies, reflect the current status of colonisation of the microbe, such as in the gut, and thus, is a useful monitor of contemporary infection. Serum antibody, on the other hand, persists for some time after the microbe is eliminated from the body. A positive serum antibody test, therefore, reflects both past and present exposure to antigen, which is less helpful to the clinician. A positive secretious antibody test, on the other hand, indicates present or contemporary infection by the microbe.

The diagnosis of H. pylori infection can be made by microscopy, microbiological culture or urease detection in gastric mucosal biopsies, urea breath test or by the presence of specific antibodies in serum ELISAs. It might be predicted that H. pylori infection, being an infection of the gastric mucosa, would elicit an IgA antibody response in gastric secretion. However it has been discovered that H. pylori-specific antibody in mucous secretion is of the IgG class and not IgA as might have been expected. Little IgA antibody, if any, is detected. Accordingly, AU-A-9067676 is directed to the detection of IgG in mucous secretion specific to H. pylori antigen and thereby provides a means of monitoring current, i.e. contemporary, infection by that microorganism in mammals. The corresponding academic publication is Witt et al, Frontiers in Mucosal Immunology 1, 693-696(1991).

The presence of IgG antibodies in the saliva of Helicobacter pylori positive patients has received some attention in the proceedings of the Annual Meetings of the American Gastroenterological Association. After the disclosure by Czinn et al of the presence of such antibodies in the 1989 proceedings, Larsen et al concluded in the May 1991 proceedings that salivary IgG levels are a practical, non-invasive marker of therapeutic response during a course of antibiotic therapy. In the April 1992 proceedings, Landes et al confirmed earlier observations and observed that measurement of salivary IgG to Helicobacter pylori is a simple non-invasive test for detecting H. pylori positive patients, especially in widespread or paediatric populations where other tests are not practical.

WO-A-9322682 discloses a convenient and reliable in vitro test for H. pylori. This test utilises an antigen preparation in a reaction with IgG antibody in a mucous secretion from a mammal being tested.

There is therefore a need to identify, isolate and thus provide novel antigens from H. pylori which can be used in diagnostic tests. These antigens should be specific, reliably purifiable, and should be characterised by good specificity and the lack of false positive results when used in such tests. In addition, they may also for the basis of a vaccine useful either for the treatment or prophylaxis of H. pylori infection.

### SUMMARY OF THE INVENTION

One object of embodiments of the invention is to provide a specific antigen of H. pylori that is reliably purifiable. Another object of embodiments of the invention is to provide antigens and test kits characterised by good specificity and alleviation of false positive results for H. pylori testing. Further objects will be readily apparent from reading this specification.

One embodiment of the invention is an isolated H. pylori antigenic protein having a molecular weight from about 50 kDa to about 65 kDa as determined using SDS-PAGE under denaturing and reducing conditions and wherein the amino terminal sequence of said isolated H. pylori antigenic protein is an amino acid sequence that has at least 60% identity to a sequence selected from the group consisting of:

| | |
|---|---|
| M V T L I N N E D D | |
| Met-Val-Thr-Leu-ILe-Asn-Asn-Glu-Asp-Asp | (SEQ ID NO:1); and |
| S V T L I N N E N N E R Y Y F E T | |
| Ser-Val-Thr-Leu-Ile-Asn-Asn-Glu-Asn-Asn-Glu-Arg-Tyr-Tyr-Phe-Glu-Thr | (SEQ ID NO:2). |

Another embodiment of the invention is a method of detecting and/or diagnosing H. pylori which comprises bringing into contact with a sample to be tested one or more antigens selected from the group consisting of an isolated H. pylori antigenic protein and antigenic fragments thereof wherein the molecular weight of said H. pylori antigenic protein is from about 50 kDa to about 65 kDa as determined using SDS-PAGE under denaturing and reducing conditions and the amino terminal sequence of said isolated H. pylori antigenic protein is an amino acid sequence that has at least 60% identity to a sequence selected from the group consisting of:

M V T L I N N B D D
Met-Val-Thr-Leu-Ile-Asn-Asn-Glu-Asp-Asp (SEQ ID NO:1); and
S V T L I N N E N N E R Y Y F E T
Ser-Val-Thr-Leu-Ile-Asn-Asn-Glu-Asn-Asn-Glu-Arg-Tyr-Tyr-Phe-Glu-Thr
(SEQ ID NO: 2); and detecting the presence of antibodies to H. pylon.

Another embodiment is a method for isolating a protein as defined in claim 1 which comprises the steps of preparing cultures of H. pylori, growing the cultures under appropriate conditions and harvesting them, followed by washing to yield a washed cell pellet; resuspending the washed cells in an appropriate buffer, followed by disruption of the cells;centrifugation to remove cell debris and obtaining the supernatant containing soluble cell proteins; subjecting the solution obtained to ion-exchange chromatography with a gradient elution, and assaying the fractions thus obtained for the presence of urease; pooling urease containing fractions and subjecting said pool to gel permeation chromatography; selecting the appropriate peak containing urease activity from said gel permeation chromatography; confirming the presence of the specific protein which is able to react with H. pylori specific antibodies and which has a molecular weight in the range 50-65 kDa in the isolate and isolating the protein; and reacting the isolated protein with an antibody that recognises the epitope of the amino acid sequence:

Met-Val-Thr-Leu-Ile-Asn-Asn-Glu (SEQ ID NO: 3).

Another embodiment of the invention is a composition capable of eliciting an immune response in a subject which comprises at least one antigen selected from the group consisting of an isolated H. pylori antigenic protein and antigenic fragments thereof wherein the protein comprises a sequence described herein. Yet another embodiment of the invention is a method for inducing an immune response in a subject comprising administering to the subject a at least one antigen selected from the group consisting of an isolated H. pylori antigenic protein and antigenic fragments thereof wherein the protein has the size characteristics and comprises a sequence as described herein.

Yet another embodiment of the invention is a method for the treatment or prophylaxis of H. pylori infection in a subject, the method comprising administering to a subject in need of such treatment an effective amount of an at least one antigen selected from the group consisting of an isolated H. pylori antigenic protein and antigenic fragments thereof wherein the protein has the size characteristics and comprises a sequence as described herein. Yet another embodiment is a diagnostic kit for detecting H. pylori infection in a subject, wherein the kit comprises an antigen as described herein. Other embodiments will be apparent from the specification.

### DESCRIPTION OF THE DRAWINGS

FIG. 1 is a plot showing antibody response against time for two rabbits inoculated with the peptide of SEQ ID NO: 3.

FIG. 2 is a Coomassie Blue stained gel obtained from native PAGE in which the protein of the present invention is compared with that obtained by the method set out in U.S. Pat. No. 5,262,156.

FIG. 3 is a silver stained gel obtained from native PAGE in which the protein of the present invention is compared with that obtained by the method set out in U.S. Pat No. 5,262,156.

FIG. 4 is a silver stained high density gel obtained from native PAGE in which the protein of the present invention is compared with that obtained by the method set out in U.S. Pat. No. 5,262,156.

### DETAILED DESCRIPTION OF THE INVENTION

The present inventors have isolated and partially characterised such an antigen and thus, in a first aspect, the present invention provides a protein being an H. pylori antigen and having a molecular weight in the range 50-65 kDa, as determined by SDS-PAGE under denaturing and reducing conditions.

Suitably, the antigenic protein has, as its amino terminal end, an amino acid sequence selected from one of the following:

| | |
|---|---|
| M V T L I N N E D D | |
| Met-Val-Thr-Leu-Ile-Asn-Asn-Glu-Asp-Asp | (SEQ ID NO:1); and |
| S V T L I N N E N N E R Y Y F E T | |
| Ser-Val-Thr-Leu-Ile-Asn-Asn-Glu-Asn-Asn-Glu-Arg-Tyr-Tyr-Phe-Glu-Thr | (SEQ ID NO:2) |

or a sequence substantially homologous thereto.

In the context of the present invention, the term “homologous” relates to proteins or polypeptides which include one or more additions, deletions, substitutions or the like when compared with the amino acid sequence of the present invention. In addition, it may be possible to replace one amino acid with another of similar “type”. For instance replacing one hydrophobic amino acid with another. One can use a program such as the CLUSTAL program to compare amino acid sequences. This program compares amino acid sequences and finds the optimal alignment by inserting spaces in either sequence as appropriate. It is possible to calculate amino acid identity or similarity (identity plus conservation of amino acid type) for an optimal alignment. A program like BLASTx will align the longest stretch of similar sequences and assign a value to the fit. It is thus possible to obtain a comparison where several regions of similarity are found, each having a different score. Both types of analysis are contemplated in the present invention.

For the purposes of the present invention, a protein sequence may be regarded as substantially homologous to another protein sequence if a significant number of the constituent amino acids exhibit homology when using the one of the algorithms mentioned above. At least 40%, 50%, 60%, 70%, 80%, 90%, 95% or even 99%, in increasing order of preference, of the amino acids, may be homologous.

The inventors have compared the sequence of the protein of the invention with that of sequences on the various data bases which are available and found that it has some similarity to a citrate synthase enzyme isolated from Rickettsia. The present inventors have therefore referred to the protein as a citrate synthase homologue. The sequence is, however, quite different from that of any protein previously isolated from H. pylori.

As mentioned above, the molecular weight range for the protein is in the range 50 to 65 kDa. However, varying values have been attributed to the molecular weight when it was measured by the inventors on different occasions. When the protein was first isolated by the inventors, the molecular weight was measured as 55-65 kDa by SDS-PAGE under denaturing and reducing conditions. However, in later experiments, the molecular weight measured by SDS-PAGE under reducing conditions was found to be 50 kDa while, under non reducing conditions it was seen as a doublet having molecular masses 50 and 52 kDa. The inventors have determined, however, that the same protein was isolated on all occasions. The variation in measured molecular weight can be attributed to the normal variation found in this method of molecular weight measurement.

It has also been found that parts of the whole protein are themselves antigenic. Thus, in a second aspect, the present invention provides an antigenic fragment of the protein of the invention.

In one preferred embodiment of this aspect of the invention, the antigenic fragment is a peptide having the sequence:

M V T L I N N E
Met-Val-Thr-Leu-Ile-Asn-Asn-Glu (SEQ ID NO:3).

The skilled man will appreciate that some variation in the sequence of this fragment will be possible, while still retaining its antigenic properties. Such variants also form part of the invention.

The antigenic protein, or fragments thereof, of the present invention can be provided alone, as a purified or isolated preparation, or as part of a mixture with other H. pylori antigenic proteins.

In a third aspect, therefore, the invention provides an antigen composition comprising a protein of the invention and one or more antigenic fragments thereof. Such a composition can be used for the detection and/or diagnosis of H. pylori. In one embodiment the composition comprises one or more additional H. pylori antigens.

In a fourth aspect, the invention provides a method of detecting and/or diagnosing H. pylori which comprises:

(a) bringing into contact with a sample to be tested one or more antigens selected from the group consisting of antigenic proteins as defined above and antigenic fragments thereof; and

(b) detecting the presence of antibodies to H. pylori.

In particular, the protein or antigenic fragment thereof of the invention or a combination thereof can be used to detect IgG antibodies. Suitably, a sample to be tested will be a biological sample, eg a sample of blood or saliva. An example of a suitable method for the detection of H. pylori using a sample of a mucous secretion is described in WO-A-9322682.

In a fifth aspect, the invention provides the use of an antigenic protein or antigenic fragment thereof of the present invention or a combination thereof in detecting and/or diagnosing H. pylori. Preferably, the detecting and/or diagnosing is carried out in vitro.

In a sixth aspect, the present invention provides a method of isolating an antigenic protein of the invention, which process comprises the following steps:

(a) preparing cultures of H. pylori, growing the cultures under appropriate conditions and harvesting them, followed by washing to yield a washed cell pellet;

(b) resuspending the washed cells in an appropriate buffer, followed by disruption of the cells;

(c) centrifugation to remove cell debris and obtaining the supernatant containing soluble cell proteins;

(d) subjecting the solution obtained to ion-exchange chromatography with a gradient elution, and assaying the fractions thus obtained for the presence of urease;

(e) pooling urease containing fractions and subjecting said pool to gel permeation chromatography;

(f) selecting the appropriate peak; and

(g) confirming the presence of the specific protein which is able to react with H. pylori specific antibodies and which has a molecular weight in the range 50-65 kDa in the isolate and isolating the protein.

The antigenic protein, antigenic fragment or combination thereof of the invention can be provided as part of a kit for use in in vitro detection and/or diagnosis of H. pylori. Thus, in a seventh aspect, the present invention provides a kit for use in the detection and/or diagnosis of H. pylori comprising one or more antigens selected from the group consisting of antigenic proteins of the present invention and fragments thereof.

In addition, the antigenic protein or antigenic fragment thereof of the invention can be used to induce an immune response against H. pylori. Thus, in a further aspect, the present invention provides a composition capable of eliciting an immune response in a subject, said composition comprising one or more antigens selected from the group consisting of the protein of the invention and antigenic fragments thereof together with a physiologically acceptable excipient. Suitably, the composition will be a vaccine composition, optionally comprising one or other suitable adjuvants. Such a vaccine composition may be either a prophylactic or therapeutic vaccine composition.

The vaccine compositions of the invention can include one or more adjuvants. Examples of adjuvants well known in the art include inorganic gels such as aluminium hydroxide or water-in-oil emulsions such as incomplete Freund's adjuvant. Other useful adjuvants will be well known to the skilled man.

In yet further aspects, the present invention provides:

(a) the use of the protein or one or more antigenic fragments thereof of the invention in the preparation of an immunogenic composition, preferably a vaccine;

(b) the use of such an immunogenic composition in inducing an immune response in a subject; and

(c) a method for the treatment or prophylaxis of H. pylori infection in a subject, which comprises the step of administering to the subject an effective amount of the protein, at least one antigenic fragment or an antigen composition of the invention, preferably as a vaccine.

Preferred features of each aspect of the invention are as for each other aspect mutatis mutandis. Each document cited is herein incorporated by reference in its entirety. Priority document U.S. Ser. No. 09/165,895 filed Sep. 30, 1998 is herein incorporated by reference in its entirety.

The invention will now be described with reference to the following examples, which should not be construed as limiting the invention in any way.

### EXAMPLE 1

(a) Cultures of H. pylori were grown under appropriate conditions and the cells harvested into phosphate buffered saline (PBS). This was followed by repeated centrifugation to remove cell debris and other contaminants, for example agar, and fresh PBS was added three times to yield a washed cell pellet.

(b) The washed cells were resuspended in 0.1 M TRIS-HCl buffer, pH 7.2 to be used in the ion exchange chromatography step. The cell suspension was then subjected to sonication (6 μ for 30 seconds, 60 seconds off, repeated 25 times for a 10 ml sample containing cells from 100 agar plates) of sufficient intensity and duration to ensure disruption of the cells.

(c) The suspension was then centrifuged to remove cell debris and the supernatant, containing soluble cell proteins, was obtained.

(d) The solution from step (c) was then subjected to fractionation by ion-exchange chromatography using strong anion exchange resin such as MonoQ™ or Q-Sepharose™ (Pharmacia), using a gradient elution based on increasing the sodium chloride concentration of the elution buffer from 0 to 1.0 M in a predetermined manner. The fractions were then assayed for the presence of urease.

(e) The urease containing fractions were then pooled and were subjected to gel permeation chromatography using a resin with a cut-off range of 5×10³ to 5×10⁶ Da for globular protein.

(f) The appropriate peak was selected by:

(i) carrying out a urease assay of all the fractions and identifying the protein peak containing the urease activity; and

(ii) analysing all the fractions shown to be urease positive and the protein containing peaks immediately adjacent to the urease peak but of lower molecular weight by spotting one microgram of protein from these fractions onto nitrocellulose or an equivalent, drying and then determining their ability to react with H. pylori specific antibodies in serum or saliva samples from H. pylori positive individuals.

(g) The presence of the specific protein in the isolate was confirmed by subjecting it to polyacrylamide gel electrophoresis (PAGE) and Western blotting, and analysing the blot using IgG from a pool prepared from human serum collected from H. pylori positive individuals. The PAGE was carried out under denaturing conditions and the protein identified as having a weight in the range 55-65 kDa was isolated.

### EXAMPLE 2

Rabbits were inoculated with the peptide M-V-T-L-J-N-N-E (SEQ ID NO: 3). Blood samples were assayed as described below to test for antibody response.

### Assay Method

To the blood samples was added 25 μl of 1% Thiomersal solution per ml of blood (i.e. a concentration of 25 μl/ml), the samples then being stored at −40° C. prior to testing.

The assays were carried out using plates wherein each well is coated with 100 μl of 5 μ/ml H. pylori antigen (antigen preparation as described in WO-A-93/22682) followed by 300 μl of 1% Byco A solution as a post coat.

The blood samples were diluted using CDL wash buffer to produce a range of dilutions.

| CDL wash buffer | quanities of 1 dm³ | |
|---|---|---|
| Tris | 12.11 | g |
| 5M HCl | 15 | ml |
| distilled water | 400 | ml |
| NaCl | 87.66 | g |
| Thiomersal | 0.1 | g |
| Tween 80 ™ | 50 | g |

(i) add Tris to distilled water;

(ii) add 5M HCl until pH 7.80 at 20° C. results;

(iii) add Thiomersal;

(iv) add NaCl;

(v) add Tween 80™;

(vi) make up final volume with distilled water.

A Swine anti-Rabbit horseradish peroxidase conjugate with ABTS (2,2-azino-di-[3-ethylbenzthiazolinsulfonate]) diluted 1/25 with Peroxidase Citrate Buffer as substrate was used to detect bound antibody, green color indicating a positive result.

| Peroxidase Citrate Buffer | quantities for 1 dm³ | |
|---|---|---|
| Distilled water | 700 | ml |
| Citric acid | 23.0 | g |
| NaOH | 100 | ml |
| 30% H₂O₂ | 0.5 | ml |

Adjust to pH 4.0 with 1M NaOH.

The assay protocol was as follows:

(i) 100 μl of diluted sample added to each well;

(ii) incubate for 30 mins (with shaking);

(iii) wash ×5 with CDL wash buffer and dry plate;

(iv) 100 μl of Swine anti-Rabbit HRP added to each well;

(v) incubate for 30 mins (with shaking);

(vi) wash ×5 and dry plate;

(vii) 100 μl of ABTS added to each well;

(viii) incubate for 30 minutes; and

(ix) read plate at 414 nm.

### Results

The peak antibody response occurred around six months post inoculation. Table 1 below shows the results over time for blood samples from two rabbits inoculated with the peptide. The data is for 1/100 diluted samples.

ABSORBANCE

| Months post inoculation | Rabbit 1 | Rabbit 2 |
|---|---|---|
| 2 | 0.484 | 0.149 |
| 3 | 0.129 | 1.154 |
| 4 | 0.491 | 1.057 |
| 5 | 1.236 | 0.7255 |
| 6 | 1.656 | 0.899 |
| 7 | 0.89 | 0.847 |
| 8 | 0.4095 | 0.7275 |

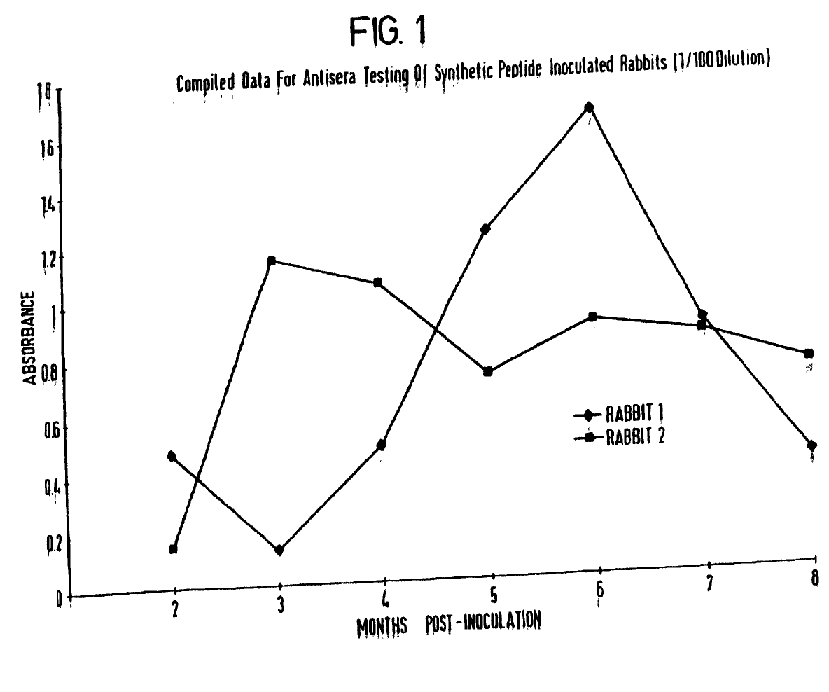

These results are shown in FIG. 1.

Clearly it can be seen that the peptide elicits an anti-H. pylori antibody response.

### EXAMPLE 3

### Use of Citrate Synthase Homologue as a Vaccine

The H. pylori antigen was prepared from a sonicate supernatant of H. pylori strain NCTC 11637 using a procedure similar to that of Example 1. The molecular weight of the required protein was measured again using SDS-PAGE under reducing conditions. This time the molecular weight was obtained as 50-52 kDa, although the protein was the same.

N-terminal sequencing was carried out at Newcastle Protein at the University of Newcastle, Newcastle, NSW, Australia and yielded an N-terminal sequence of

S V T L I N N E D D E R Y Y
Ser-Val-Thr-Leu-Ile-Asn-Asn-Glu-Asn-Asn-Glu-Arg-Tyr-Tyr.

This protein was identified by BLAST analysis, using the SwisspProt database as the H. pylori citrate synthase homologue. There was no detectable amount of urease activity in the purified protein preparation.

### Immunisation of Mice

Female specific pathogen free C57BL/6 mice were obtained from the Central Animal House at the University of Newcastle, NSW, Australia. Mice were immunised by the intra-Peyer's patch (IPP) route to test the efficacy of the antigen as a vaccine candidate as this immunisation route has been shown to give maximal intestinal immunisation (Dunkley et al, Immunology 57, 379-385 (1986), Cripps et al, Infect. Immun. 62, 1427-1436 (1994)) and is therefore useful for screening proteins which can be used in oral vaccines. Antigen (at 0.5 mg protein ml⁻¹) was contained in an homogenate of equal quantities of PBS and Freunds incomplete adjuvant. For IPP immunisation, each mouse was anaesthetised by intraperitoneal injection of 200 μl of a ketamine (Parnell Laboratories, Australia), xylazine (Bayer) mixture made by mixing 10 ml of ketamine (100 μg/ml) and 1 ml of xylazine (100 μg/ml), the abdomen shaved and swabbed with 70% alcohol and a midline incision made in the skin and muscle layers to expose the intestine. Visible Peyer's patches were located along the length of the intestine and approximately 3 μl of homogenate injected directly under the serosa of each Peyer's patch. The muscle and skin layers were sutured and the mouse kept warm until recovery from anaesthesia. For each experiment, 10 mice were immunised and another 10 mice left untreated as the unimmunised controls.

### Infection of Mice with H. pylori

Mice were infected two weeks after immunisation. H. pylori Sydney strain 1 (SS1) was obtained from Prof. A Lee, The University of New South Wales, Sydney, Australia. This strain of H. pylori has been shown to successfully colonise the stomachs of C57BL/6 mice. The H. pylori was grown on chocolate agar plates for 3 days in a microaerophilic 37° C. incubator and harvested into PBS. The concentration of H. pylori was determined from the optical density reading at 405 nm and a regression curve relating optical density to H. pylori concentration. Mice were infected by gavage on three successive days with a 100μl volume containing approximately 10⁸ H. pylori, and actual concentration of live H. pylori was determined by culture of serial 10-fold dilutions of the live H. pylori preparation on chocolate agar for 3 days. The actual dose of live H. pylori was therefore calculated retrospectively. The doses on the three successive days were: Experiment 1:4.5×10⁸, 4.25×10⁸, 4.25×10⁸; and Experiment 2:1.5×10⁸, 1.0×10⁸, 8.75×10⁸. This is a total of 10⁹ Experiment 1 and 1.125×10⁹ for Experiment 2.

### Sample Collection

Four weeks after infection, the mice were killed by intraperitoneal pentobarbital overdose and the stomachs removed. The stomachs were cut in half longitudinally and one half was homogenised in 1 ml of PBS and aliquots of serial dilutions plated out on chocolate agar plates and cultured for 3 days. Colonies were counted to determine the number of colony-forming units (CFU) of H. pylori in the stomach of each mouse. The mean ±SEM was calculated for each group. Only mice showing detectable infection were included in this calculation as the infection did not establish in all mice. This absence of infection occurred in both the unimmunised and the immunised groups.

### Results

The pooled data from two experiments in which mice were pre-immunised IPP with the 50/52 kDa protein are shown in Table 2. In Experiment 1, significant (P,0.05) bacterial clearance (91% reduction in CFU) was observed in the immunised group compared to the unimmunised group. In Experiment 2, significant (P,0.01) bacterial clearance (84% reduction in CFU) was observed in the immunised group compared to the unimmunised group.

TABLE 2

| | | | | H. pylori |
|---|---|---|---|---|
| | | | | cleared |
| | | | Live bacteria | (% of |
| | | Mouse | per stomach | immunised |
| Experiment | No. Infected | Group | (10⁴ CFU) | group) |
| 1 | 10/12 | Unimmunised | 36 ± 7 | |
| | | (10) | | |
| 1 | 8/9 | Immunised | 3.2 ± 1.7 | 91 |
| | | (8) | | |
| 2 | 9/10 | Unimmunised | 40 ± 9 | |
| | | (9) | | |
| 2 | 11/11 | immunised | 6.6 ± 3.6 | 84 |
| | | (11) | | |

The protein of the present invention is likely to be useful as a vaccine antigen as it is present in significant quantities in the H. pylori strain NCTC 11637 and is easily purified. This example demonstrates that the protein is a potent vaccine antigen when used to immunise mice by IPP injection. This suggests that this protein will be an appropriate antigen for inclusion in a human oral vaccine.

### EXAMPLE 4

### Use of Native and Recombinant Protein as a Vaccine

The Experiment described in Example 3 was repeated using three groups of mice. Group A was an unimmunised control, Group B was treated with the protein obtained in the method of Example 1 and Group C was treated with a recombinant version of this protein. The results of the Experiment are set out in Table 3.

TABLE 3

| | | Live bacteria per |
|---|---|---|
| Mouse Group | No. of Mice | stomach (10⁴ CFU) |
| A | 10 | 36.15 ± 29 |
| B | 11 | 6.63 ± 11.8 |
| C | 10 | 5.26 ± 4.6 |

This Example demonstrates that the recombinant protein is just as effective when used as a vaccine as the native protein.

### EXAMPLE 5

### Comparison of the Protein of the Invention with H. pylori Derived Proteins of Similar Molecular Weight

The protein of the present invention prepared as set out in Example 1 was compared with a protein extracted from H. pylori by the method described by Alemohammad in U.S. Pat. No. 5,262,156.

The major difference between the method described by Alemohammad and that of the present invention is that Alemohammad extracted the proteins from the crude H. pylori extract using octyl-β,D-glucopyranoside whereas the present inventors have used an aqueous buffer as described in Example 1.

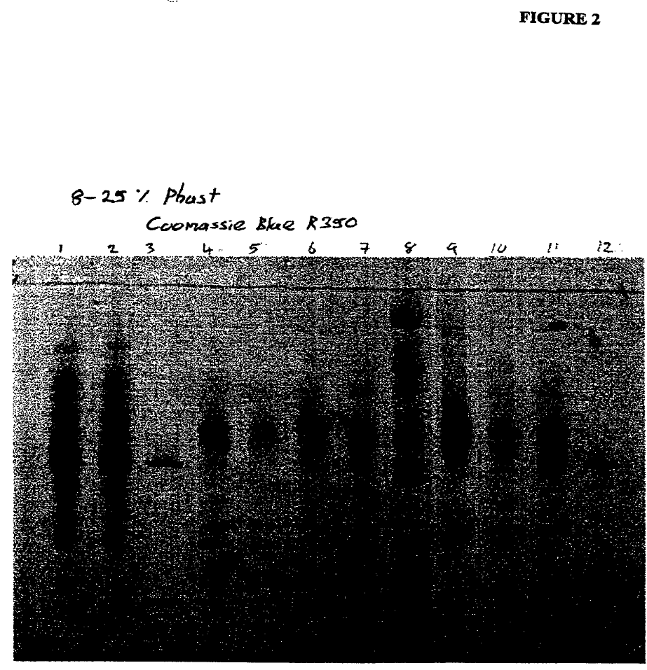

The protein of the present invention and the mixture obtained by the method of Alemohammad were analysed by native PAGE (8-25% gradient gel) and the results are shown in FIG. 2, which is a Coomassie Blue R350 stained gel with lanes numbered 1 to 12.

Lanes 1 and 2 are unpurified sonicate.

Lanes 3 and 12 are the protein of the present invention.

Lane 4 is a sample taken after the first PBS wash of H. pylori cells grown on plates.

Lane 5 is a sample taken after the second PBS wash of H. pylori cells grown on plates.

Lane 6 is an octyl-β,D-glucopyranoside extract of the cells after the second PBS wash and filtered through a 0.2 μm filter.

Lane 7 is as for Lane 6 but not filtered.

Lane 8 is a molecular weight marker

Lane 9 is a sample taken after the first PBS wash of H. pylori cells grown in a fermenter.

Lane 10 is an octyl-β,D-glucopyranoside extract of the cells from the fermenter after the second PBS wash and filtered through a 0.2 μm filter.

Lane 11 is as for lane 10 but not filtered.

Thus, the samples run in lanes 3 and 12 are the protein of the present invention. Those in Lanes 6 and 10 are the protein prepared by the process of Alemohammad and the samples run in Lanes 4, 5, 7, 9 and 11 are samples taken during the extraction process described by Alemohammad. Lanes 6 and 7 were samples taken from H. pylori cells grown on agar plates while Lanes 10 and 11 were samples taken from H. pylori cells grown in a fermenter. However there was no difference in the material obtained by these two methods.

In contrast, however, all of the samples prepared using the extraction method described by Alemohammad are completely different from the protein samples prepared according to the present invention. Thus, the inventors have concluded that they have extracted a new protein, not one of the proteins described by Alemohammad.

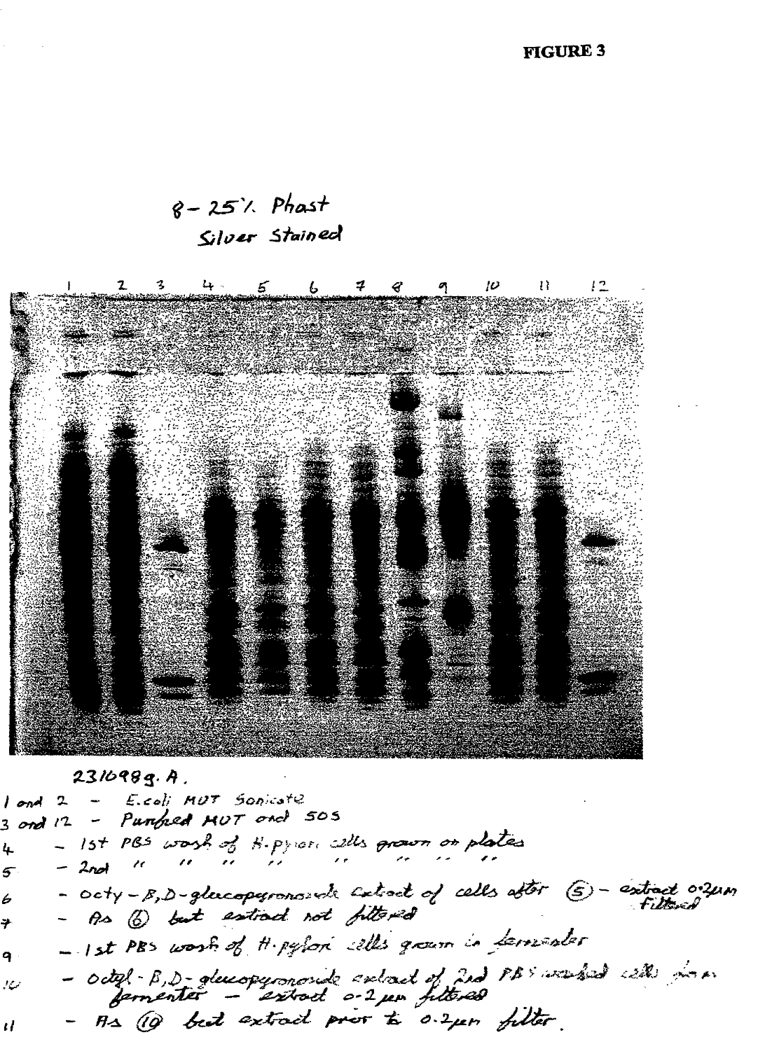

The experiment was repeated and the same materials were run on the same gel. However, this time the gel was stained with silver, which is more sensitive than Coomassie Blue. The results are shown in FIG. 3, in which the lanes are the same as those in FIG. 2. The results were the same as for the previous experiment and confirmed that the protein of the present invention is completely different from that described in U.S. Pat. No. 5,262,156.

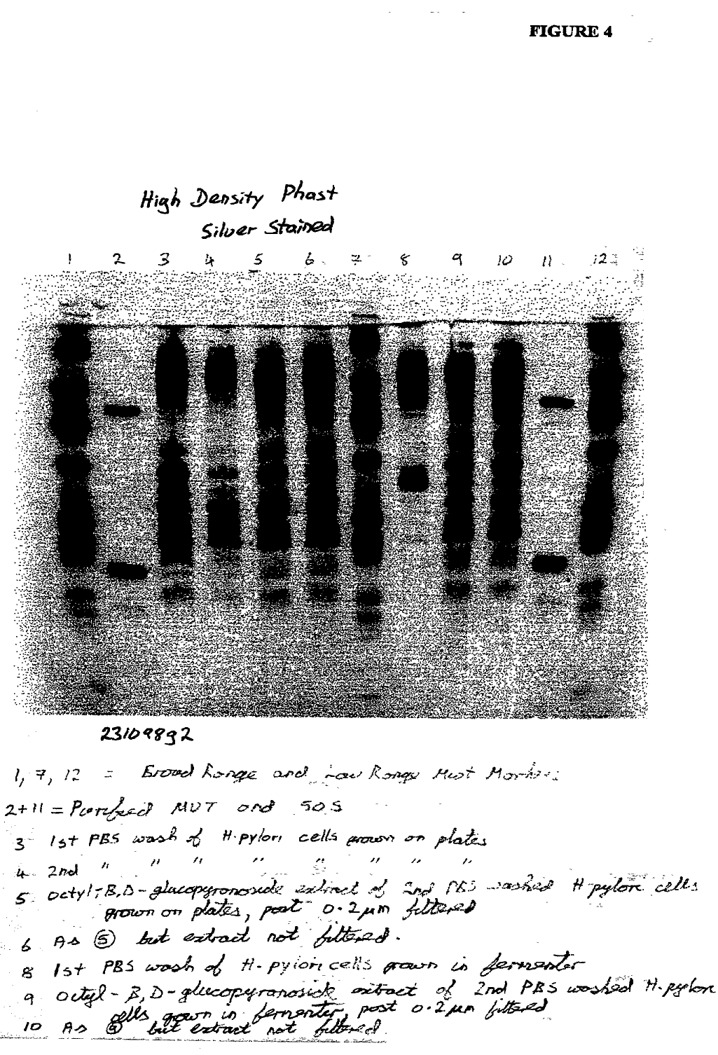

Finally, in order to obtain further confirmation of these results, the materials were run on a high density gel (8-25% Pharmacia High Density Phast Gel) with greater sensitivity for the lower molecular weight materials. The results from this are shown in FIG. 4. In this Figure:

Lane 1 is a molecular weight marker.

Lane 2 is the protein of the present invention.

Lane 3 is a sample taken after the first PBS wash of H. pylori cells grown on plates.

Lane 4 is a sample taken after the second PBS wash of H. pylori cells grown on plates.

Lane 5 is an octyl-β,D-glucopyranoside extract of the cells after the second PBS wash and filtered through a 0.2 μm filter.

Lane 6 is as for Lane 5 but not filtered.

Lane 7 is a molecular weight marker.

Lane 8 is a sample taken after the first PBS wash of H. pylori cells grown in a fermenter.

Lane 9 is an octyl-β,D-glucopyranoside extract of the cells from the fermenter after the second PBS wash and filtered through a 0.2 μm filter.

Lane 10 is as for lane 9 but not filtered.

Lane 11 is the protein of the present invention.

Lane 12 is a molecular weight marker.

Once again, the results of the experiment confirmed that the protein of the present invention is completely different from that disclosed in U.S. Pat. No. 5,262,156.

1. An isolated H. pylori antigenic protein wherein:
(a) the molecular weight of said isolated H. pylori antigenic protein is from about 50 kDa to about 65 kDa as determined using SDS-PAGE under denaturing and reducing conditions; and
(b) the amino terminal sequence of said isolated H. pylori antigenic protein is an amino acid sequence that has at least 60% identity to a sequence selected from the group consisting of:

| | |
|---|---|
| M V T L I N N E D D | |
| Met-Val-Thr-Leu-Ile-Asn-Asn-Glu-Asp-Asp | (SEQ ID NO:1); and |
| S V T L I N N E N N E R Y Y F E T | |
| Ser-Val-Thr-Leu-Ile-Asn-Asn-Glu-Asn-Asn-Glu-Arg-Tyr-Tyr-Phe-Glu-Thr | (SEQ ID NO:2). |

2. The isolated H. pylori antigenic protein of claim 1 wherein the amino terminal sequence is Met-Val-Thr-Leu-Ile-Asn-Asn-Glu-Asp-Asp (SEQ ID NO: 1).

3. The isolated H. pylori antigenic protein of claim 1 wherein the amino terminal sequence is Ser-Val-Thr-Leu-Ile-Asn-Asn-Glu-Asn-Asn-Glu-Arg-Tyr-Tyr-Phe-Glu-Thr (SEQ ID NO: 2).

4. An antigenic fragment of a protein according to claim 1.

5. An antigenic fragment according to claim 4 which has the sequence:
Met-Val-Thr-Leu-Ile-Asn-Asn-Glu (SEQ ID NO: 3).

6. A method of detecting and/or diagnosing H. pylori which comprises:
(a) bringing into contact with a sample to be tested one or more antigens selected from the group consisting of an isolated H. pylori antigenic protein and antigenic fragments thereof wherein:
(i) the molecular weight of said H. pylori antigenic protein is from about 50 kDa to about 65 kDa as determined using SDS-PAGE under denaturing and reducing conditions; and
(ii) the amino terminal sequence of said isolated H. pylori antigenic protein is an amino acid sequence that has at least 60% identity to a sequence selected from the group consisting of:

| | |
|---|---|
| M V T L I N N E D D | |
| Met-Val-Thr-Leu-Ile-Asn-Asn-Glu-Asp-Asp | (SEQ ID NO:1); and |
| S V T L I N N E N N E R Y Y F E T | |
| Ser-Val-Thr-Leu-Ile-Asn-Asn-Glu-Asn-Asn-Glu-Arg-Tyr-Tyr-Phe-Glu-Thr | (SEQ ID NO:2); and |

(b) detecting the presence of antibodies to H. pylori.

7. The method of claim 6 wherein said H. pylori antigenic protein has the amino terminal sequence:
Met-Val-Thr-Leu-Ile-Asn-Asn-Glu-Asp-Asp (SEQ ID NO: 1).

8. The method of claim 6 wherein said H. pylori antigenic protein has the amino terminal sequence:
Ser-Val-Thr-Leu-Ile-Asn-Asn-Glu-Asn-Asn-Glu-Arg-Tyr-Tyr-Phe-Glu-Thr (SEQ ID NO: 2).

9. The method of claim 7 wherein the fragment has the sequence:
Met-Val-Thr-Leu-Ile-Asn-Asn-Glu (SEQ ID NO: 3).

10. The method of claim 6 wherein the sample is a sample of saliva.

11. The method of claim 6 wherein the detecting and/or diagnosing is carried out in vitro.

12. A method for isolating a protein as defined in claim 1 which comprises the steps of:
(a) preparing cultures of H. pylori, growing the cultures under appropriate conditions and harvesting them, followed by washing to yield a washed cell pellet;
(b) resuspending the washed cells in an appropriate buffer, followed by disruption of the cells;
(c) centrifugation to remove cell debris and obtaining the supernatant containing soluble cell proteins;
(d) subjecting the solution obtained to ion-exchange chromatography with a gradient elution, and assaying the fractions thus obtained for the presence of urease;
(e) pooling urease containing fractions and subjecting said pool to gel permeation chromatography;
(f) selecting the appropriate peak containing urease activity from said gel permeation chromatography;
(g) confirming the presence of the specific protein which is able to react with H. pylori specific antibodies and which has a molecular weight in the range 50-65 kDa in the isolate and isolating the protein; and
(h) reacting the isolated protein with an antibody that recognises the epitope of the amino acid sequence:
Met-Val-Thr-Leu-Ile-Asn-Asn-Glu (SEQ ID NO: 3).

13. A kit for use in the detection and/or diagnosis of H. pylori which comprises At least one antigen selected from the group consisting of an isolated H. pylori antigenic protein and antigenic fragments thereof wherein:
(i) the molecular weight of said H. pylori antigenic protein is from about 50 kDa to about 65 kDa as determined using SDS-PAGE under denaturing and reducing conditions; and
(ii) the amino terminal sequence of said isolated H. pylori antigenic protein is an amino acid sequence that has at least 60% identity to a sequence selected from the group consisting of:

| | |
|---|---|
| M V T L I N N E D D | |
| Met-Val-Thr-Leu-Ile-Asn-Asn-Glu-Asp-Asp | (SEQ ID NO:1); and |
| S V T L I N N E N N E R Y Y F E T | |
| Ser-Val-Thr-Leu-Ile-Asn-Asn-Glu-Asn-Asn-Glu-Arg-Tyr-Tyr-Phe-Glu-Thr | (SEQ ID NO:2). |

14. A composition capable of eliciting an immune response in a subject which comprises at least one antigen selected from the group consisting of an isolated H. pylori antigenic protein and antigenic fragments thereof wherein:
(i) the molecular weight of said H. pylori antigenic protein is from about 50 kDa to about 65 kDa as determined using SDS-PAGE under denaturing and reducing conditions; and
(ii) the amino terminal sequence of said isolated H. pylori antigenic protein is an amino acid sequence that has at least 60% identity to a sequence selected from the group consisting of:

| | |
|---|---|
| MV T L I N N B D D | |
| Met-Val-Thr-Leu-Ile-Asn-Asn-Glu-Asp-Asp | (SEQ ID NO:1); and |
| S V T L I N N E N N E R Y Y F E T | |
| Ser-Val-Thr-Leu-Ile-Asn-Asn-Glu-Asn-Asn-Glu-Arg-Tyr-Tyr-Phe-Glu-Thr | (SEQ ID NO:2); |

together with a pharmaceutically acceptable excipient.

15. The composition of claim 14 which is a vaccine composition and which further comprises one or more adjuvants.

16. A method for inducing an immune response in a subject comprising administering to the subject a at least one antigen selected from the group consisting of an isolated H. pylori antigenic protein and antigenic fragments thereof wherein:
(i) the molecular weight of said H. pylori antigenic protein is from about 50 kDa to about 65 kDa as determined using SDS-PAGE under denaturing and reducing conditions; and
(ii) the amino terminal sequence of said isolated H. pylori antigenic protein is an amino acid sequence that has at least 60% identity to a sequence selected from the group consisting of:
```
              M V T L I N N E D D
            Met-Val-Thr-Leu-Ile-Asn-Asn-Glu-Asp-Asp (SEQ ID NO:1); and
              S V T L I N N E N N E R Y Y F E T
Ser-Val-Thr-Leu-Ile-Asn-Asn-Glu-Asn-Asn-Glu-Arg-Tyr-Tyr-Phe-Glu-Thr (SEQ ID NO:2).
```

17. A method for the treatment or prophylaxis of H. pylori infection in a subject, the method comprising administering to a subject in need of such treatment an effective amount of an at least one antigen selected from the group consisting of an isolated H. pylori antigenic protein and antigenic fragments thereof wherein:
(i) the molecular weight of said H. pylori antigenic protein is from about 50 kDa to about 65 kDa as determined using SDS-PAGE under denaturing and reducing conditions; and
(ii) the amino terminal sequence of said isolated H. pylori antigenic protein is an amino acid sequence that has at least 60% identity to a sequence selected from the group consisting of:
```
                M V T L I N N E D D
              Met-Val-Thr-Leu-Ile-Asn-Asn-Glu-Asp-Asp (SEQ ID NO:1); and
              S V T L I N N E N N E R Y Y F E T
Ser-Val-Thr-Leu-Ile-Asn-Asn-Glu-Asn-Asn-Glu-Arg-Tyr-Tyr-Phe-Glu-Thr (SEQ ID NO:2).
```

18. A method as claimed in claim 17 wherein said at least one antigen is administered in the form of a vaccine.


SEQUENCE LISTING (4 sequences)

SEQ ID NO: 1 - protein, 10 aa, Helicobacter pylori
MVTLINNEDD

SEQ ID NO: 2 - protein, 17 aa, Helicobacter pylori
SVTLINNENNERYYFET

SEQ ID NO: 3 - protein, 8 aa, Helicobacter pylori
MVTLINNE

SEQ ID NO: 4 - protein, 14 aa, Helicobacter pylori
SVTLINNENNERYY